(19) United States
(12) Patent Application Publication
(10) Pub. No.: US 2026/0224856 A1
(43) Pub. Date: Aug. 6, 2026

(54) Guide Sheaths and Methods for Transeptally Delivering an Instrument to or Beyond the Left Ventricular Outflow Tract of a Subject

(71) Applicant: The Regents of the University of California, Oakland, CA (US)

(72) Inventor: Gregory Marcus, San Francisco, CA (US)

(21) Appl. No.: 19/151,169

(22) PCT Filed: Jan. 26, 2024
(86) PCT No.: PCT/US2024/013135

§ 371 Date: Jul. 25, 2025

Related U.S. Application Data
(60) Provisional application No. 63/441,668, filed on Jan. 27, 2023.

(51) Int. Cl. A61M 25/06; A61B 18/00; A61B 18/14; A61F 2/962; A61M 5/00; A61M 25/10

(52) U.S. Cl. CPC A61M 25/0662; A61B 18/1492; A61F 2/962; A61M 5/007; A61M 25/104; A61B 2018/00351; A61B 2018/00577

(57) ABSTRACT

Methods of transeptally delivering an instrument to or beyond the left ventricular outflow tract of a subject via a guide sheath are provided. Guide sheaths for use in the disclosed methods include an elongate structure comprising a channel for receiving the instrument running therethrough from a proximal end to a distal end. The elongate structure of the subject invention includes a first portion which is a fixed curve portion or a deflectable portion, and a second portion which is a fixed curve portion or a deflectable portion. First and second portions of interest are adapted for transeptally delivering the instrument to or beyond the left ventricular outflow tract of the subject. Guide sheaths and kits for practicing methods of the invention are also provided.

### CROSS-REFERENCE TO RELATED APPLICATIONS

This application claims the benefit of U.S. Provisional Patent Application No. 63/441,668, filed Jan. 27, 2023, which application is incorporated herein by reference in its entirety.

### INTRODUCTION

Catheters have been in use for medical procedures for many years. For example, catheters have been used to convey an electrical stimulus to a selected location within the human body. Another use is monitoring and making measurements for diagnostic tests within the human body. Catheters are also used by physicians to examine, diagnose and treat while positioned at a specific location within the body which is otherwise inaccessible without more invasive procedures. In use, catheters are inserted into a major vein or artery which is near the body surface. These catheters are then guided to the specific location for examination, diagnosis or treatment by manipulating the catheter through the artery or vein of the human body.

Catheters are used increasingly for medical procedures involving the human heart. In these procedures, a catheter is typically advanced to the heart through veins or arteries and then is positioned at a specified location within the heart. Typically, the catheter is inserted in an artery or vein in the leg, neck, upper chest or arm of the patient and threaded, generally with the aid of a guidewire and conventional introducer, through various arteries or veins until the tip of the catheter reaches the desired location in the heart.

The heart is primarily composed of multiple fibers which are responsible for the propagation of signals necessary for normal, electrical and mechanical function. The presence of an arrhythmogenic site or abnormal pathway which may bypass or short circuit the normal conducting fibers in the heart often causes abnormally rapid rhythms of the heart, which are referred to as tachycardias. Tachycardias may be defined as ventricular tachycardias (VTs) and supraventricular tachycardias (SVTs). VTs originate in the left or right ventricle, and are typically caused by arrhythmogenic sites associated with ventricular myocardial disease. SVTs originate in the atria or the atrioventricular (AV) junction and are frequently caused by abnormal circuits or foci.

Electrophysiological catheter ablation procedures are often employed to treat premature ventricular contractions and ventricular tachycardia. Such procedures often involve destructive energy to the target tissue including direct current electrical energy, radiofrequency electrical energy, microwave energy laser energy, cryoenergy, ultrasound and the like. In such procedures, a catheter having a suitable electrode is introduced to the appropriate heart chamber and manipulated so that the electrode lies proximate the target tissue. The electrode can then be employed to ablate a region of the tissue which forms part of the arrhythmogenic site or the abnormal pathway.

Some procedures, most notably angioplasty procedures, utilize a catheter which often contains an inflatable balloon secured to the catheter. In some of these medical procedures, the catheter contains a pair of inflatable balloons used to limit the portion of the vessel that is treated and/or to assure that the catheter remains at a fixed location within the vessel throughout the medical procedure. For example, as a method for resolving atherosclerotic plaque build-up, stenotic lesions are now routinely opened by the use of balloon angioplasty. In this procedure, a balloon-carrying catheter is navigated through the patient's vascular system to the location of the stenosis. The balloon is inflated by fluid injected through a lumen of the catheter to apply pressure to the walls of the clogged vessel, thereby opening it.

However, heart catheterization procedures have been hampered by the inability of the operator to maneuver the catheter tip to the precise location of the relevant tissue. This problem is most pronounced in the ventricular chambers because of the high degree of trabeculation. Many medical practitioners performing a catheter-based intervention in and around the left ventricle perform procedures via a “retrograde approach”. This approach involves introducing the catheter into the femoral artery, advancing it up through the aorta and into the left ventricle. Procedures using a retrograde approach are described in, e.g., U.S. Pat. Nos. 5,640,955 and 5,722,400.

### SUMMARY

The present inventor has realized that conventional methods for delivering catheters to left ventricular locations suffer from procedural deficiencies. In particular, it was found that that left ventricular outflow tract, and particularly coronary cusp interventions generally require a retrograde aortic approach, although a transseptal approach (i.e., puncturing the atrial septum) would otherwise be preferred. It has been hypothesized that retrograde aortic approaches may be associated with negative outcomes such as the production of cerebral emboli. Accordingly, methods for transeptally delivering an instrument to or beyond the left ventricular outflow tract of the subject are desirable. The disclosed methods, guide sheaths and kits of the invention satisfy this desire.

Aspects of the invention include methods of transeptally delivering an instrument to or beyond the left ventricular outflow tract of a subject. Methods of interest include delivering the instrument via a guide sheath comprising an elongate structure comprising a channel for receiving the instrument running therethrough from a proximal end to a distal end. Elongate structures of interest for the subject methods include a first portion which is a fixed curve portion or a deflectable portion, and a second portion which is a fixed curve portion or a deflectable portion. The first and second portions are adapted for transeptally delivering the instrument to or beyond the left ventricular outflow tract of the subject. In some embodiments, the second portion is distal to the first portion. In some such embodiments, the first portion is a fixed curve portion. In alternative embodiments, the first portion is a deflectable portion. In some versions, the second portion is a deflectable portion. The first and second portions may be separated from each other by a distance ranging from, e.g., 0 cm to 50 cm. In certain cases, the first portion begins at a location along the elongate structure that is at a distance from the distal end ranging from 11 mm to 40 cm. In select versions, the first portion ends at a location along the elongate structure that is at a distance from the distal end ranging from 1 cm to 40 cm. In certain embodiments, the first portion is curved or deflectable to curve at an angle ranging from 5° to 355°. In certain cases, the second portion begins at a location along the elongate structure that is at a distance from the distal end ranging from 1 mm to 40 cm. In some versions, the second portion ends at a location along the elongate structure that is at a distance from the distal end ranging from 0 cm to 39 cm. In select instances, the second portion is curved or deflectable to curve at an angle ranging from 5° to 355°.

The instrument delivered by the subject methods may, in certain embodiments, be a catheter (e.g., an ablation catheter). In some such embodiments, methods include delivering the ablation catheter to a coronary cusp of the subject, and ablating the coronary cusp using the ablation catheter. Methods according to certain instances include treating premature ventricular contractions (PVCs) or ventricular tachycardia of the subject. In certain versions, methods also include delivering the instrument to the coronary arteries of the subject. In some such versions, methods include injecting contrast dye into the coronary arteries of the subject. Angioplasty balloons, stents and/or other devices for expand the lumen of a coronary artery may also be introduced to the coronary artery using the instrument. Methods according to some embodiments may include delivering the instrument to the aortic valve of the subject (e.g., to deliver a replacement aortic valve to the subject).

Aspects of the invention also include guide sheaths. The subject guide sheaths include an elongate structure comprising a channel for receiving an instrument running therethrough from a proximal end to a distal end. Elongate structures of interest include a first portion which is a fixed curve portion or a deflectable portion, and a second portion which is a fixed curve portion or a deflectable portion. The first and second portions are adapted for transeptally delivering the instrument to or beyond the left ventricular outflow tract of the subject. In some embodiments, the second portion is distal to the first portion. In some such embodiments, the first portion is a fixed curve portion. In alternative embodiments, the first portion is a deflectable portion. In some versions, the second portion is a deflectable portion. The first and second portions may be separated from each other by a distance ranging from, e.g., 0 cm to 50 cm. In certain cases, the first portion begins at a location along the elongate structure that is at a distance from the distal end ranging from 11 mm to 40 cm. In select versions, the first portion ends at a location along the elongate structure that is at a distance from the distal end ranging from 1 cm to 40 cm. In certain embodiments, the first portion is curved or deflectable to curve at an angle ranging from 5° to 355°. In certain cases, the second portion begins at a location along the elongate structure that is at a distance from the distal end ranging from 1 mm to 40 cm. In some versions, the second portion ends at a location along the elongate structure that is at a distance from the distal end ranging from 0 cm to 39 cm. In select instances, the second portion is curved or deflectable to curve at an angle ranging from 5° to 355°.

Aspects of the invention additionally include kits, where kits include the guide sheath of the invention (e.g., described above). Kits may additionally include one or more of a catheter (e.g., balloon catheter, ablation catheter), a balloon, a catheter introducer, mechanical transseptal needle or wire (e.g., a radio frequency transeptal needle or wire).

### BRIEF DESCRIPTION OF THE FIGURES

The invention is best understood from the following detailed description when read in conjunction with the accompanying drawings. It is emphasized that, according to common practice, the various features of the drawings are not to-scale. On the contrary, the dimensions of the various features are arbitrarily expanded or reduced for clarity. Included in the drawings are the following figures:

FIG. 1 depicts the advancement of a guide sheath of the invention through the inferior vena cava according to embodiments of the disclosed methods.

FIG. 2 depicts a transseptal puncture and advancement of a guide sheath through the mitral valve of a subject according to embodiments of the disclosed methods.

FIG. 3A-3B depict the advancement of a guide sheath through the left ventricular outflow tract and aortic valve (FIG. 3A), as well as the extension of a catheter to ablate tissue associated with the coronary cusp (FIG. 3B).

FIG. 4 depicts the advancement of the guide sheath to the left coronary artery.

FIG. 5A-5C depict embodiments of a guide sheath having a deflectable portion at a distal location (FIG. 5A), at a more proximal location (FIG. 5B), and at both locations (FIG. 5C).

### DETAILED DESCRIPTION

Methods of transeptally delivering an instrument to or beyond the left ventricular outflow tract of a subject via a guide sheath are provided. Guide sheaths for use in the disclosed methods include an elongate structure comprising a channel for receiving the instrument running therethrough from a proximal end to a distal end. The elongate structure of the subject invention includes a first portion which is a fixed curve portion or a deflectable portion, and a second portion which is a fixed curve portion or a deflectable portion. First and second portions of interest are adapted for transeptally delivering the instrument to or beyond the left ventricular outflow tract of the subject. Guide sheaths and kits for practicing methods of the invention are also provided.

Before the guide sheaths, methods and kits of the present disclosure are described in greater detail, it is to be understood that the methods, systems and computer-readable media not limited to particular embodiments described, as such may, of course, vary. It is also to be understood that the terminology used herein is for the purpose of describing particular embodiments only, and is not intended to be limiting, since the scope of the methods, systems and computer-readable media will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the methods, systems and computer-readable media. The upper and lower limits of these smaller ranges may independently be included in the smaller ranges and are also encompassed within the methods, systems and computer-readable media, subject to any specifically excluded limit in the stated range. Where the stated range includes one or both of the limits, ranges excluding either or both of those included limits are also included in the methods, systems and computer-readable media.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Unless defined otherwise, all technical and scientific terms used herein have the same meaning as commonly understood by one of ordinary skill in the art to which the methods, systems and computer-readable media belong. Although any methods, systems and computer-readable media similar or equivalent to those described herein can also be used in the practice or testing of the methods, systems and computer-readable media, representative illustrative methods, systems and computer-readable media are now described.

All publications and patents cited in this specification are herein incorporated by reference as if each individual publication or patent were specifically and individually indicated to be incorporated by reference and are incorporated herein by reference to disclose and describe the materials and/or methods in connection with which the publications are cited. The citation of any publication is for its disclosure prior to the filing date and should not be construed as an admission that the present methods, systems and computer-readable media are not entitled to antedate such publication, as the date of publication provided may be different from the actual publication date which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

It is appreciated that certain features of the methods, systems and computer-readable media, which are, for clarity, described in the context of separate embodiments, may also be provided in combination in a single embodiment. Conversely, various features of the methods, systems and computer-readable media, which are, for brevity, described in the context of a single embodiment, may also be provided separately or in any suitable sub-combination. All combinations of the embodiments are specifically embraced by the present disclosure and are disclosed herein just as if each and every combination was individually and explicitly disclosed, to the extent that such combinations embrace operable processes and/or compositions. In addition, all sub-combinations listed in the embodiments describing such variables are also specifically embraced by the present methods, systems and computer-readable media and are disclosed herein just as if each and every such sub-combination was individually and explicitly disclosed herein.

As will be apparent to those of skill in the art upon reading this disclosure, each of the individual embodiments described and illustrated herein has discrete components and features which may be readily separated from or combined with the features of any of the other several embodiments without departing from the scope or spirit of the present methods, systems and computer-readable media. Any recited method can be carried out in the order of events recited or in any other order that is logically possible.

### Methods

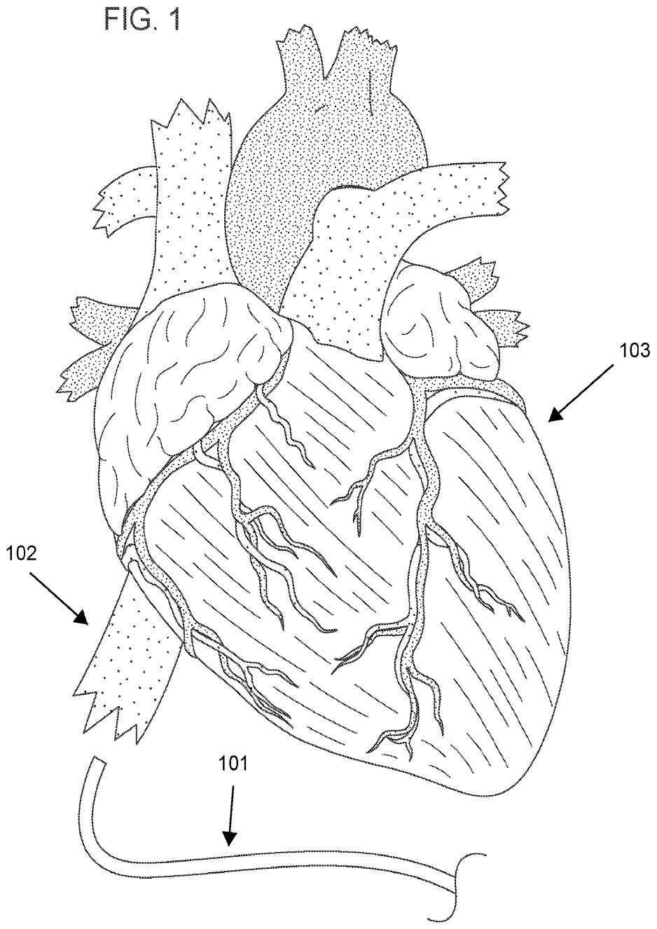

As discussed above, aspects of the invention include transeptally delivering an instrument to or beyond the left ventricular outflow tract of a subject. The term “transseptal delivery”, also referred to herein as a “transseptal approach”, is used in its conventional sense to refer to a technique for catheter positioning that involves passing the catheter through the interatrial septum between the right and left atria. Generally, a transseptal approach involves introducing a sheath apparatus into the right femoral vein of the subject, and advancing the sheath through the inferior vena cava into the right atrium (see, e.g., FIG. 1). When in the right atrium, the interatrial septum is punctured, e.g., via a needle.

Guide sheaths that may be employed include an elongate structure comprising a channel for receiving the instrument running therethrough from a proximal end to a distal end. The terms “proximal” and “distal” end are used in their conventional sense to refer to near to the point of origin and far from the point of origin. In the present case, the distal end is the end of the guide sheath that is configured to be employed in the heart, while the proximal end is the end configured to be nearest the point of entry (e.g., incision into the femoral vein). Lengths of the elongate structures may vary. For example, the elongate structure of the subject guide sheaths may range from 70 cm to 120 cm, such as 80 cm to 100 cm, and including 85 cm to 95 cm. The elongate structures may also have any suitable outer diameter. In some embodiments, outer diameters range from 0.5 mm to 1.5 mm, such as 0.6 mm to 1.2 mm, such as 0.7 mm to 0.9 mm, and including 0.75 mm to 0.85 mm. The elongate structures may additionally have any suitable inner diameter. Inner diameters of interest range from 0.5 mm to 1.5 mm, such as 0.6 mm to 1.2 mm, such as 0.7 mm to 0.9 mm and including 0.75 mm to 0.85 mm. The guide sheaths of the invention may be comprised of any convenient material. In some cases, guide sheaths are comprised of a polymeric material (e.g., a plastic material). Polymeric materials of interest include, but are not limited to, polypropylene, polycarbonate, polyethylene, polyamide, polyvinyl chloride (PVC), acrylonitrile butadiene styrene (ABS), combinations thereof, and the like. In certain embodiments, the guide sheaths are comprised of a metal. Metals of interest may include, but are not limited to, stainless steel, titanium, chromium, cobalt, nickel, and the like, as well as alloys thereof. In select embodiments, methods include transeptally delivering guide sheaths that include one or more surface coatings. In some cases, the coating is a hydrophobic coating. Surface coatings that may be employed include, but are not limited to, heparin, EDTA, oxalate, combinations thereof, and the like.

The guide sheaths for use in the subject methods include first and second portions that are adapted for transeptally delivering the instrument to or beyond the left ventricular outflow tract of the subject. Each of the first and second portions may be a fixed curve portion or a deflectable portion. By “fixed curve portion”, it is meant that the relevant portion of the guide has a pre-formed shape. In certain cases where a force is applied to a fixed curve portion that distorts the fixed curve shape, said fixed curve portion is configured such that it re-adopts the pre-formed shape when the force is withdrawn (i.e., and not some other, different, shape). In other cases, the fixed curve portion is configured such that the portion will not change in shape when an external force is applied (i.e., where the force does not break the portion). The fixed curve portion of the guide sheath may, in certain cases, be curved at an angle ranging from 5° to 355°, such as 10° to 200°, such as 20° to 150°, and including 50° to 100°. On the other hand, a “deflectable” portion is capable of being adjusted such that the portion may adopt any one of many different configurations or angles, as desired. The deflectable portion may be deflected by any suitable means. In some embodiments, methods include deflecting the deflectable portion using a deflection control wire. The deflection control wire may, in some embodiments, control the deflection of the tip in a plane parallel to the guide sheath (vertical deflection). The deflection control wire may, in certain cases, be comprised of stainless steel. In embodiments, tension in the wire may cause the portion to deflect. In certain cases, rotation of the guide sheath may be achieved by a torqueable wire. The proximal portion of the torqueable wire may be more rigid relative to the distal portion. The deflectable portion of the guide sheath may, in certain cases, be deflectable to curve at an angle ranging from 5° to 355° such as 10° to 200°, such as 20° to 150°, and including 50° to 100°.

The first and second portions may be positioned at multiple suitable locations along the elongate structure of the guide sheath. In some cases, the second portion is distal to the first portion. The distance (i.e., along the length of the elongate structure) separating the first and second portions may vary. In some cases, the first portion is separated by the second portion by a distance of from 0 cm to 50 cm, such as 0 cm to 5 cm, such as 5 cm to 10 cm, such as 10 cm to 15 cm, such as 15 cm to 20 cm, such as 20 cm to 25 cm, such as 25 cm to 30 cm, such as 30 cm to 35 cm, such as 35 cm to 40 cm, such as 40 cm to 45 cm, and including 45 cm to 50 cm. In some cases where the first and second portions are separated from each other by a distance of 0 cm, the first and second portions may be considered adjacent to one another. In other words, in such cases, there is no intermediate portion of the elongate structure separating the first and second portions.

The location of the first and second portions with respect to the elongate structure may vary. The first and second portions may, in certain cases, be described as having a beginning and ending, where the beginning is proximal to the ending along the length of the elongate structure. The location of the beginning and ending of the first and second portions may be defined, e.g., with respect to a distance along the elongate structure measured from the distal end. In select instances, the first portion begins at a location along the elongate structure that is at a distance from the distal end ranging from 11 mm to 40 cm, such as 11 mm to 50 mm, such as 50 mm to 100 mm, such as 100 mm to 150 mm, such as 150 mm to 200 mm, such as 200 mm to 250 mm, such as 250 mm to 500 mm, such as 500 mm to 750 mm, such as 750 mm to 1 cm, such as 1 cm to 5 cm, such as 5 cm to 10 cm, such as 10 cm to 15 cm, such as 15 cm to 20 cm, such as 20 cm to 25 cm, such as 25 cm to 30 cm, such as 30 cm to 35 cm, and including 35 cm to 40 cm. In select instances, the first portion ends at a location along the elongate structure that is at a distance from the distal end ranging from 1 cm to 40 cm, such as 1 cm to 5 cm, such as 5 cm to 10 cm, such as 10 cm to 15 cm, such as 15 cm to 20 cm, such as 20 cm to 25 cm, such as 25 cm to 30 cm, such as 30 cm to 35 cm, and including 35 cm to 40 cm. Similarly, in some cases, the second portion begins at a location along the elongate structure that is at a distance from the distal end ranging from 1 mm to 40 cm, such as 1 mm to 5 mm, such as 5 mm to 10 mm, such as 10 mm to 50 mm, such as 50 mm to 100 mm, such as 100 mm to 150 mm, such as 150 mm to 200 mm, such as 200 mm to 250 mm, such as 250 mm to 500 mm, such as 500 mm to 750 mm, such as 750 mm to 1 cm, such as 1 cm to 5 cm, such as 5 cm to 10 cm, such as 10 cm to 15 cm, such as 15 cm to 20 cm, such as 20 cm to 25 cm, such as 25 cm to 30 cm, such as 30 cm to 35 cm, and including 35 cm to 40 cm. In some versions, the second portion ends at a location along the elongate structure that is at a distance from the distal end ranging from 0 mm to 39 cm, such as 0 mm to 1 mm, such as 1 mm to 5 mm, such as 5 mm to 10 mm, such as 10 mm to 50 mm, such as 50 mm to 100 mm, such as 100 mm to 150 mm, such as 150 mm to 200 mm, such as 200 mm to 250 mm, such as 250 mm to 500 mm, such as 500 mm to 750 mm, such as 750 mm to 1 cm, such as 1 cm to 5 cm, such as 5 cm to 10 cm, such as 10 cm to 15 cm, such as 15 cm to 20 cm, such as 20 cm to 25 cm, such as 25 cm to 30 cm, such as 30 cm to 35 cm, and including 35 cm to 39 cm.

As discussed above, each of the first and second portions of the guide sheath may either be a fixed portion or a deflectable portion. In some cases, both of the first and second portions are fixed portions. In other cases, both of the first and second portions are deflectable portions. In still other cases, the first portion is a deflectable portion, and the second portion is a fixed portion. In yet other cases, the first portion is a fixed portion, and the second portion is a deflectable portion. In select embodiments, guide sheaths may comprise additional fixed and/or deflectable portions. In some such embodiments, the elongate structure includes a third portion. The third portion may be either a fixed portion or a deflectable portion, and may be located at any convenient position relative to the aforementioned first and second portions. For example, the third portion could be positioned at a location that is proximal relative to the first and second portions, between the first and second portions, or distal to the first and second portions. Guide sheaths having more than 3 portions (e.g., a fourth portion, a fifth portion, etc.) are also envisioned.

As discussed above, methods of the invention include transeptally delivering an instrument to or beyond the left ventricular outflow tract of a subject. By “to or beyond” the left ventricular outflow tract, it is meant that the instrument could be delivered to a location within the left ventricular outflow tract, adjacent to it, or at a location that is through the aortic valve (i.e., “beyond” the left ventricular outflow tract). Such methods generally include inserting the guide sheath into the femoral vein and guiding it through the inferior vena cava until the tip of the catheter reaches the superior vena cava. For example, FIG. 1 depicts guide sheath 101 being advanced through vena cava 102 into heart 103.

Subsequently, the guide sheath is navigated to the right atrium of the heart, and to the interatrial septum separating the left and right atria where the transseptal puncture is carried out. While the puncture site may be selected from multiple different sites depending on the requirements of a given procedure, methods according to some embodiments of the invention include carrying out the transseptal puncture at the fossa ovalis. In some cases, methods include employing a needle to carry out the puncture. Needles that can be adapted for use in the subject methods may vary. In some instances, the needle is a curved needle (e.g., a Brockenbrough curved needle). Once the sheath is placed against the puncture site, methods include advancing the needle distally through the sheath until it punctures the puncture site. Transseptal puncture needles that may be adapted for use in the subject methods are described in, e.g., U.S. Pat. Nos. 7,635,353; 8,114,110; 10,426,515; 11,457,903; and 11,523,808; as well as U.S. Patent Application Publication Nos. 2015/0265344; 2017/0065297; and 2022/0080161; the disclosures of which are herein incorporated by reference in their entirety. In some cases, a dilator is employed to complete the puncture, i.e., to generate a hole in the interatrial septum that is sufficient for the sheath to pass through. Dilators that may be adapted for use in the subject methods may be found in, e.g., International Application Publication No. WO2019/196293. Dilators according to certain embodiments include a tubular body with a piercing structure at the distal end. In alternative embodiments, methods include puncturing the interatrial septum via a trocar. In select cases, methods include inserting a stylet into the guide sheath in order to stiffen the needle as it passes. The stylet may include, in some cases, a flexible rod.

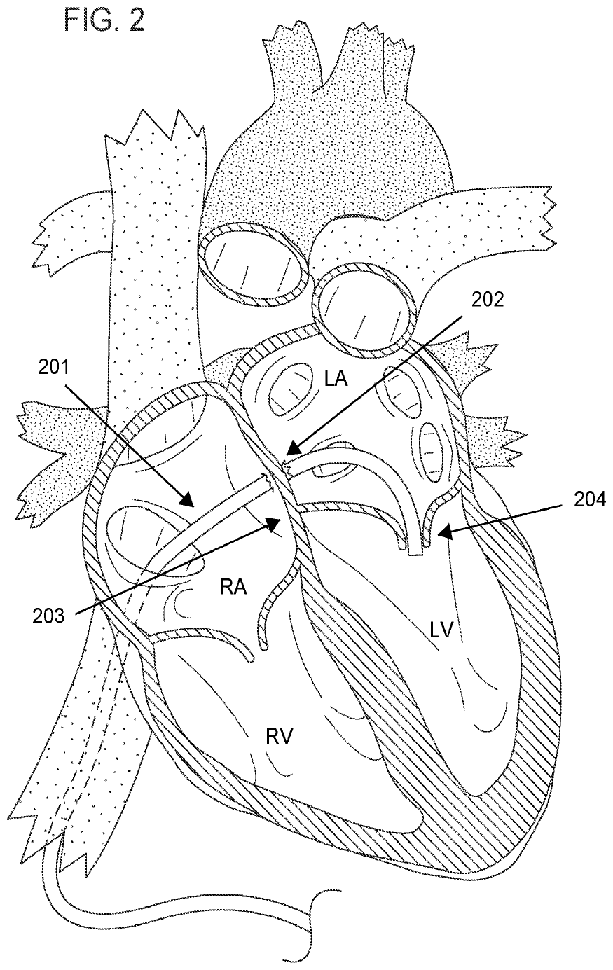

Following the transseptal puncture, the guide sheath is advanced through the left atrium. Once in the left atrium, the guide sheath is advanced through the mitral valve into the left ventricle. As shown in FIG. 2, guide sheath 201 has been advanced through the right atrium (RA) through puncture site 202 in interatrial septum 203, into the left atrium (LA), and through the mitral valve 204 in to the left ventricle (LV).

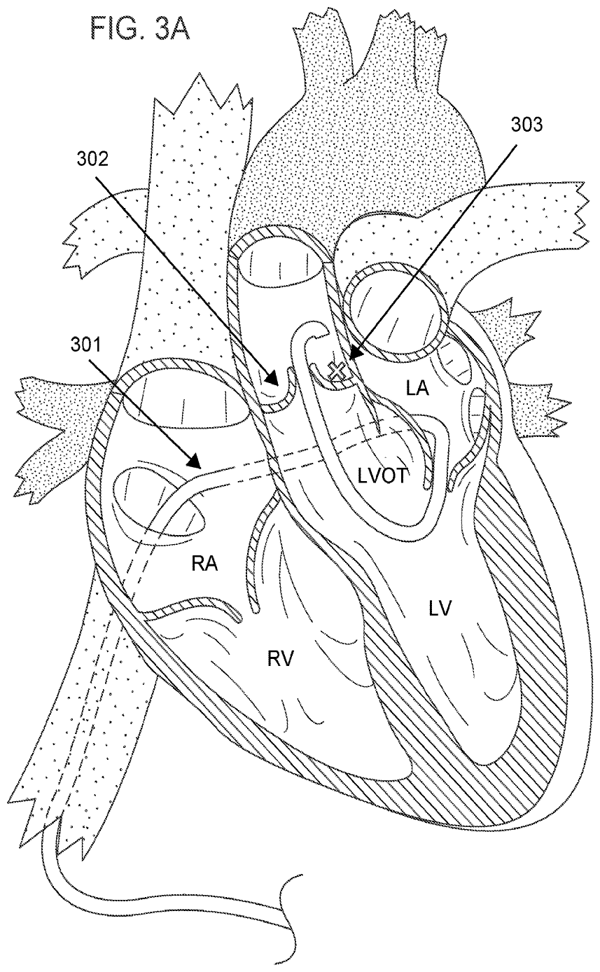

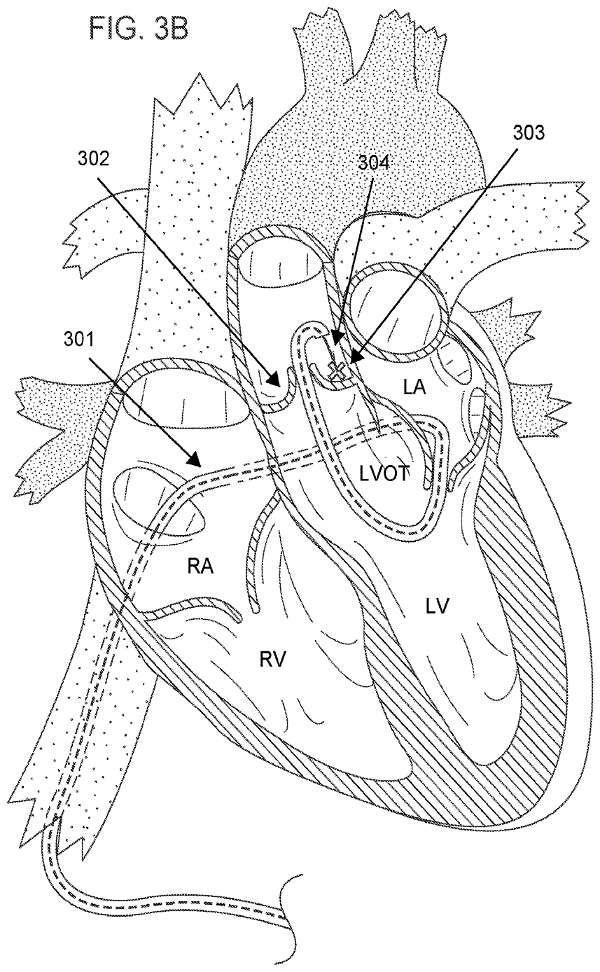

Once the guide sheath has been advanced into the left ventricle, methods include advancing the guide sheath to or beyond the left ventricular outflow tract. As is known in the art, the left ventricular outflow tract describes a portion of the left ventricle through which blood passes to reach the aorta. In some cases, the left ventricular outflow tract may also be referred to as the “aortic vestibule”. In some versions, methods include advancing the guide sheath into the ventricular outflow tract without passing through the aortic valve. In other versions, methods include advancing the guide sheath through the aortic valve and to a location that is beyond the left ventricular outflow tract. In some embodiments, methods include advancing the guide sheath into the ascending aorta of the subject. In certain cases, methods include advancing the guide sheath to the coronary cusp associated with the aortic valve. As shown in FIG. 3A, guide sheath 301 has been advanced through the left atrium (LA) into the left ventricle (LV), into the left ventricular outflow tract (LVOT), and through the aortic valve 302 in order to reach point of interest 303. The structure of guide sheath 301 (discussed in detail herein) allows it to reach point of interest 303 via a transseptal approach rather than a retrograde approach.

Any suitable instrument may be employed in the transseptal delivery described above. In some instances, the instrument is sized to be fed through the guide sheath such that the instrument reaches the location at or beyond the left ventricular outflow tract of a subject. In some cases, the instrument is a catheter. A catheter is a medical tool that has been used for many years. They are employed, e.g., for medical procedures to examine, diagnose, and treat while positioned at a specific location within the body that is otherwise inaccessible without more invasive procedures. Catheters that may be employed in the subject methods may vary depending on the type of procedure being performed.

In some cases where the instrument includes a catheter, the catheter is an ablation catheter. As discussed above, ablation involves lesioning dysfunctional cardiac tissue to treat a subject for a condition. Ablation can also be performed to remove undesired tissue such as cancer cells. Ablation procedures may further involve the modification of the tissue without removal, such as to stop electrical propagation through the tissue in patients with an arrhythmia. Often the ablation is performed by passing energy, such as electrical energy, through one or more electrodes causing the tissue in contact with the electrodes to heats up to an ablative temperature. Ablation procedures can be performed on patients with atrial fibrillation by ablating tissue in the heart. The ablation catheter may be configured to lesion the tissue via any convenient technique. Exemplary catheters employ radio frequency (RF) energy, to heat the tissue above 55°C., thereby creating the desired lesion. Examples of RF catheter ablation systems which deliver the RF energy as a point source using a needle-like or tip electrode fixed to the distal end of the catheter are shown in e.g., U.S. Pat. Nos. 5,083,565, 5,281,218 and 5,403,311; the disclosures of which are incorporated by reference herein in their entirety. Ablation catheters of interest also include fluid cooled RF ablation catheters, such as those disclosed in U.S. Pat. Nos. 5,334,193, 5,348,554, 5,423,811, 5,431,649 and 5,462,521; the disclosures of which are incorporated by reference herein in their entirety. Additional catheters that may be employed include those that achieve ablation using microwave technology, such as those described in U.S. Patent Application Publication No. 2021/0186608; the disclosure of which is incorporated by reference herein in its entirety. Where methods include an ablation, any convenient location in or beyond the left ventricular outflow tract may be ablated. In some such cases, the method comprises delivering the ablation catheter to a coronary cusp of the subject and ablating the coronary cusp using the ablation catheter.

FIG. 3B depicts the delivery of an instrument according to embodiments of the subject methods. As shown in FIG. 3B, a catheter 304 is passed through guide sheath 301 to point of interest 303 on the coronary cusp 302 of the subject. In the example of FIG. 3B, catheter 304 is an ablation catheter employed to ablate/lesion point of interest 303 on the coronary cusp 302 of the subject.

In some cases, the instrument comprises a balloon. In such cases, the catheter may be referred to as a “balloon catheter”, and comprises an inflatable balloon at the distal end that may be employed to enlarge a passage within the heart (e.g., so that a procedure may be carried out in the passage). The balloon may be inflated by a user for affixing it in place, for expanding a blocked vessel, for placing treatment means (e.g., stent) and/or for delivering surgical tools to a desired site. Alternatively, the balloon may be used for retrieval of objects such as stents. Any suitable balloon catheter may be employed in the subject methods. Exemplary balloon catheters that may be adapted for use in the methods of the invention are described in, e.g., U.S. Pat. Nos. 5,281,200; 6,142,973; 7,198,632; 7,316,709; 7,431,714; 7,513,886; 7.658,744; 7,708,753; 7,906,066; 8,372,034; 8,617,104; 9,005,163; 9,005,165; 9,044,571; 9,079,000; 9,192,747; 9,327,101; 9,956,384 10,124,153; 10,569,062; 10,940,294; and 11,202,884; the disclosures of which are incorporated by reference herein in their entirety.

In some embodiments, methods include delivering the instrument to the aortic valve of the subject. The aortic valve is susceptible to complications such as aortic stenosis or aortic insufficiency. One method for treating such conditions involves the use of a prosthetic valve implanted within a native aortic valve. These prosthetic valves can be implanted using various techniques, including various transcatheter techniques. In some cases where the instrument comprises a balloon, methods of the invention may include delivering the balloon to the aortic valve of the subject. In some embodiments, the method also comprises delivering a replacement aortic valve to the subject. In some such cases, a transcatheter heart valve (THV) is crimped on the end portion of a flexible and/or steerable catheter, advanced through the blood vessels following the heart to an implantation site in the heart, and then, for example, the THV can be expanded to a functional size, such as by inflating the balloon.

Methods according to some embodiments also include stent delivery. As is understood in the art, a stent is a mesh tube configured to hold open a passageway. In some such embodiments, a catheter designed for stent delivery may be employed (e.g., a stent catheter). Further details regarding catheters and stents that may be adapted for use in the subject methods may be found in, e.g., U.S. Pat. Nos. 5,261,878; 5,368,566; 7,393,358; 7,695,508; 7,879,387; and 9,283,099; the disclosures of which are incorporated by reference herein in their entirety. In select embodiments, the method comprises delivering a stent to the ascending aorta of the subject. In other cases, methods include delivering a stent into a coronary artery of the subject.

In some cases, methods include transeptally delivering an instrument to or beyond the left ventricular outflow tract of a subject to perform angiography. As is known in the art, an angiogram involves X-ray imaging of regions of blood vessels and/or the heart that have been exposed to contrast dye. Further details regarding angiograms may be found in, e.g., U.S. Pat. Nos. 9,014,453 and 10,895,623; the disclosures of which are herein incorporated by reference in their entirety. Accordingly, methods according to these embodiments of the invention include injecting contrast dye into the subject.

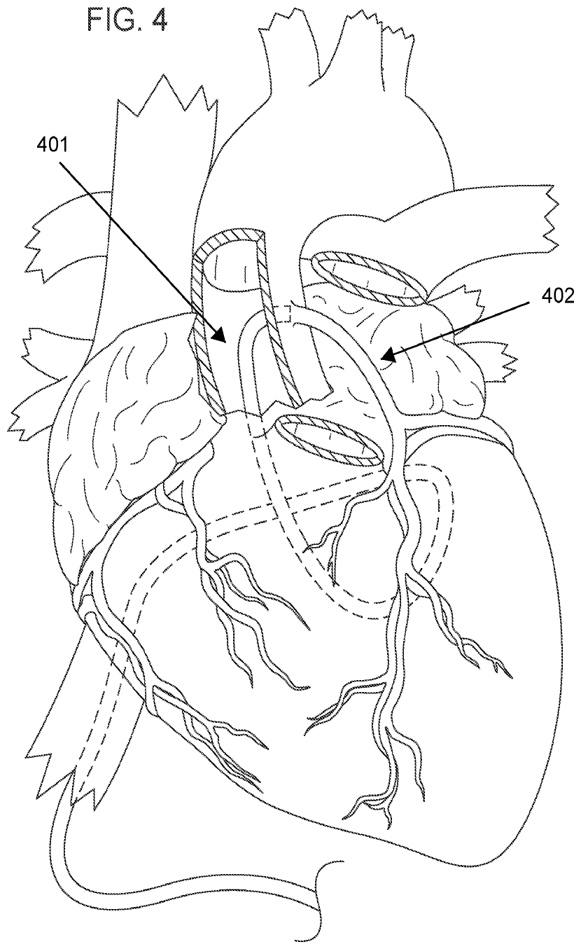

Methods according to some embodiments of the invention include delivering the instrument to the coronary arteries of the subject (e.g., the left coronary artery). Methods according to such embodiments include injecting contrast dye into the coronary arteries of the subject, i.e., to perform an angiogram (e.g., as discussed above). In some cases, methods involve performing an angioplasty. As is understood in the art, an angioplasty includes opening a coronary artery that has been blocked by coronary artery disease. In some such cases, methods include delivering an angioplasty balloon and/or device used to open and/or expand the lumen of a coronary artery into a coronary artery of the subject. Any suitable balloon and/or balloon catheter may be employed (e.g., such as those described above). In some embodiments involving stent placement, methods include delivering a stent into a coronary artery of the subject. FIG. 4 depicts the advancement of the guide sheath to the left coronary artery. As shown in FIG. 4, guide sheath 401 is advanced to left coronary artery 402. Depending on the type of procedure being performed, the guide sheath may be employed to perform an angioplasty, stent placement, angiogram, or the like. The structure of guide sheath 401 (discussed in detail herein) allows it to reach coronary artery 402 via a transseptal approach as opposed to a retrograde approach.

In some embodiments, methods include delivering the instrument to or beyond the left ventricular outflow tract of a subject via the guide sheath using an introducer. As is understood in the art, introducers are guide wires configured to advance an instrument (e.g., catheter) through a guide sheath. Introducers may be comprised of, e.g., stainless steel.

The methods of the invention may be employed to diagnose and treat a variety of different conditions. Conditions of interest include, but are not limited to atherosclerosis, cardiomyopathy, congenital heart disease, heart failure and heart valve disease. In some cases, the condition treated by the subject methods is premature ventricular contractions (PVCs). In certain instances, the condition is ventricular tachycardia. The subject treated by the subject methods may be a mammal, such as a human.

### Guide Sheaths

As discussed above, methods of the invention additionally include guide sheaths. Guide sheaths of interest include an elongate structure comprising a channel for receiving an instrument running therethrough from a proximal end to a distal end. The elongate structure includes a first portion which is a fixed curve portion or a deflectable portion, and a second portion which is a fixed curve portion or a deflectable portion. The first and second portions are adapted for transeptally delivering the instrument to or beyond the left ventricular outflow tract of the subject.

Lengths of the elongate structures may vary. For example, the elongate structure of the subject guide sheaths may range from 70 cm to 120 cm, such as 80 cm to 100 cm, and including 85 cm to 95 cm. The elongate structures may also have any suitable outer diameter. In some embodiments, outer diameters range from 0.5 mm to 1.5 mm, such as 0.6 mm to 1.2 mm, such as 0.7 mm to 0.9 mm and including 0.75 mm to 0.85 mm. The elongate structures may additionally have any suitable inner diameter. Inner diameters of interest range from 0.5 mm to 1.5 mm, such as 0.6 mm to 1.2 mm, such as 0.7 mm to 0.9 mm and including 0.75 mm to 0.85 mm. The guide sheaths of the invention may be comprised of any convenient material. In some cases, guide sheaths are comprised of a polymeric material (e.g., a plastic material). Polymeric materials of interest include, but are not limited to, polypropylene, polycarbonate, polyethylene, polyamide, polyvinyl chloride (PVC), acrylonitrile butadiene styrene (ABS), combinations thereof, and the like. In certain embodiments, the guide sheaths are comprised of a metal. Metals of interest may include, but are not limited to, stainless steel, titanium, chromium, cobalt, nickel, and the like, as well as alloys thereof. In select embodiments, guide sheaths include one or more surface coatings. In some cases, the coating is a hydrophobic coating. Surface coatings that may be employed include, but are not limited to, heparin, EDTA, oxalate, combinations thereof, and the like

The fixed curve portion of the guide sheath may, in certain cases, be curved at an angle ranging from 5° to 355°, such as 10° to 200°, such as 20° to 150°, and including 50° to 100°. The deflectable portion may be deflected by any suitable means. The deflectable portion of the guide sheath may, in certain cases, be deflectable to curve at an angle ranging from 5° to 355° such as 10° to 200°, such as 20° to 150°, and including 50° to 100°.

The first and second portions may be positioned at multiple suitable locations along the elongate structure of the guide sheath. In some cases, the second portion is distal to the first portion. The distance (i.e., along the length of the elongate structure) separating the first and second portions may vary. In some cases, the first portion is separated by the second portion by a distance of from 0 cm to 50 cm, such as 0 cm to 5 cm, such as 5 cm to 10 cm, such as 10 cm to 15 cm, such as 15 cm to 20 cm, such as 20 cm to 25 cm, such as 25 cm to 30 cm, such as 30 cm to 35 cm, such as 35 cm to 40 cm, such as 40 cm to 45 cm, and including 45 cm to 50 cm. In some cases where the first and second portions are separated from each other by a distance of 0 cm, the first and second portions may be considered adjacent to one another. In other words, in such cases, there is no intermediate portion of the elongate structure separating the first and second portions.

The location of the first and second portions with respect to the elongate structure may vary. The first and second portions may, in certain cases, be described as having a beginning and ending, where the beginning is proximal to the ending along the length of the elongate structure. The location of the beginning and ending of the first and second portions may be defined, e.g., with respect to a distance along the elongate structure measured from the distal end. In select instances, the first portion begins at a location along the elongate structure that is at a distance from the distal end ranging from 11 mm to 40 cm, such as 11 mm to 50 mm, such as 50 mm to 100 mm, such as 100 mm to 150 mm, such as 150 mm to 200 mm, such as 200 mm to 250 mm, such as 250 mm to 500 mm, such as 500 mm to 750 mm, such as 750 mm to 1 cm, such as 1 cm to 5 cm, such as 5 cm to 10 cm, such as 10 cm to 15 cm, such as 15 cm to 20 cm, such as 20 cm to 25 cm, such as 25 cm to 30 cm, such as 30 cm to 35 cm, and including 35 cm to 40 cm. In select instances, the first portion ends at a location along the elongate structure that is at a distance from the distal end ranging from 1 cm to 40 cm, such as 1 cm to 5 cm, such as 5 cm to 10 cm, such as 10 cm to 15 cm, such as 15 cm to 20 cm, such as 20 cm to 25 cm, such as 25 cm to 30 cm, such as 30 cm to 35 cm, and including 35 cm to 40 cm.

Similarly, in some cases, the second portion begins at a location along the elongate structure that is at a distance from the distal end ranging from 1 mm to 40 cm, such as 1 mm to 5 mm, such as 5 mm to 10 mm, such as 10 mm to 50 mm, such as 50 mm to 100 mm, such as 100 mm to 150 mm, such as 150 mm to 200 mm, such as 200 mm to 250 mm, such as 250 mm to 500 mm, such as 500 mm to 750 mm, such as 750 mm to 1 cm, such as 1 cm to 5 cm, such as 5 cm to 10 cm, such as 10 cm to 15 cm, such as 15 cm to 20 cm, such as 20 cm to 25 cm, such as 25 cm to 30 cm, such as 30 cm to 35 cm, and including 35 cm to 40 cm. In some versions, the second portion ends at a location along the elongate structure that is at a distance from the distal end ranging from 0 mm to 39 cm, such as 0 mm to 1 mm, such as 1 mm to 5 mm, such as 5 mm to 10 mm, such as 10 mm to 50 mm, such as 50 mm to 100 mm, such as 100 mm to 150 mm, such as 150 mm to 200 mm, such as 200 mm to 250 mm, such as 250 mm to 500 mm, such as 500 mm to 750 mm, such as 750 mm to 1 cm, such as 1 cm to 5 cm, such as 5 cm to 10 cm, such as 10 cm to 15 cm, such as 15 cm to 20 cm, such as 20 cm to 25 cm, such as 25 cm to 30 cm, such as 30 cm to 35 cm, and including 35 cm to 39 cm.

As discussed above, each of the first and second portions of the guide sheath may either be a fixed portion or a deflectable portion. In some cases, both of the first and second portions are fixed portions. In other cases, both of the first and second portions are deflectable portions. In still other cases, the first portion is a deflectable portion, and the second portion is a fixed portion. In yet other cases, the first portion is a fixed portion, and the second portion is a deflectable portion. In select embodiments, guide sheaths may comprise additional fixed and/or deflectable portions. In some such embodiments, the elongate structure includes a third portion. The third portion may be either a fixed portion or a deflectable portion, and may be located at any convenient position relative to the aforementioned first and second portions. For example, the third portion could be positioned at a location that is proximal relative to the first and second portions, between the first and second portions, or distal to the first and second portions. Guide sheaths having more than 3 portions (e.g., a fourth portion, a fifth portion, etc.) are also envisioned.

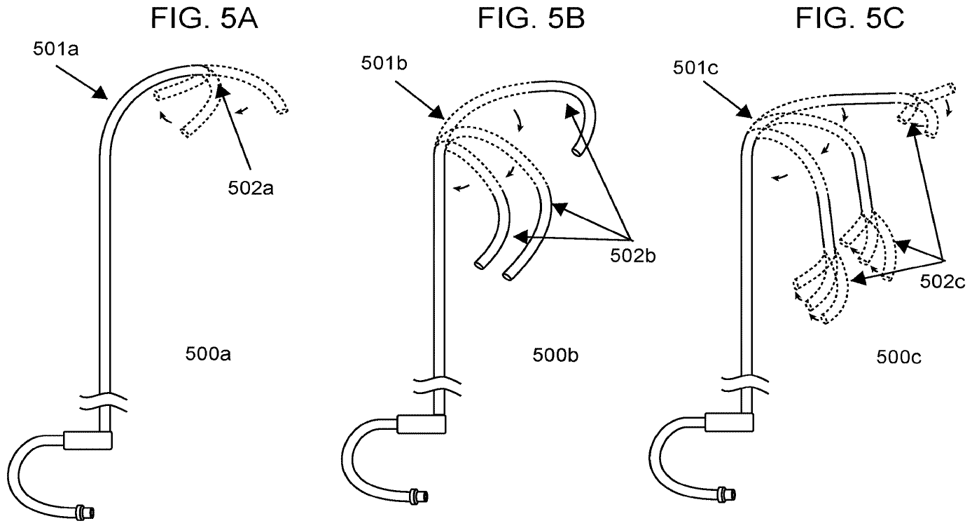

FIG. 5A-C depict embodiments of a guide sheath having a deflectable portion at different locations. Guide sheaths 500a-c include first portions 501a-c and second portions 502a-c. In FIG. 5A, the first portion 501a of guide sheath 500a is a fixed curve portion, while second portion 502a is a deflectable portion. In FIG. 5B, the first portion 501b of guide sheath 500b is a deflectable portion, while second portion 502b is a fixed curve portion. In FIG. 5C, both first portion 501c and second portion 502c of guide sheath 500c are deflectable portions.

### Kits

Aspects of the invention additionally include kits. Kits of interest include one or more guide sheaths of the invention. As discussed above, the subject guide sheaths include an elongate structure comprising a channel for receiving an instrument running therethrough from a proximal end to a distal end. The elongate structure includes a first portion which is a fixed curve portion or a deflectable portion, and a second portion which is a fixed curve portion or a deflectable portion. In addition, the first and second portions are adapted for transeptally delivering the instrument to or beyond the left ventricular outflow tract of the subject.

Kits of the invention may additionally include instructions for using the guide sheath to transeptally deliver an instrument to or beyond the left ventricular outflow tract of a subject. Instructions may be included on any suitable medium. These instructions may be present in the subject kits in a variety of forms, one or more of which may be present in the kit. One form in which these instructions may be present is as printed information on a suitable medium or substrate, e.g., a piece or pieces of paper on which the information is printed, in the packaging of the kit, in a package insert, and the like. Yet another form of these instructions is a computer readable medium, e.g., diskette, compact disk (CD), portable flash drive, and the like, on which the information has been recorded. Yet another form of these instructions that may be present is a website address which may be used via the internet to access the information at a removed site.

The subject kits may also include one or more components used for performing the methods of the invention. For example, in some embodiments, kits include one or more catheters. Any suitable cardiac catheter may be employed in the subject methods. In some cases, the catheter is an ablation catheter. The ablation catheter may be configured to lesion the tissue via any convenient technique. Exemplary catheters employ radio frequency (RF) energy. Ablation catheters of interest also include fluid cooled RF ablation catheters. Additional catheters that may be employed include those that achieve ablation using microwave technology. In additional embodiments, the catheter is a balloon catheter. As discussed above, balloon catheters comprise an inflatable balloon at the distal end that may be employed to enlarge a passage within the heart (e.g., so that a procedure may be carried out in the passage). The balloon may be inflated by a user for affixing it in place, for expanding a blocked vessel, for placing treatment means (e.g., stent) and/or for delivering surgical tools to a desired site. Alternatively, the balloon may be used for retrieval of objects such as stents. Kits according to some embodiments also include one or more balloons for use with a balloon catheter.

In some embodiments, kits include one or more introducers. As discussed above, introducers are guide wires configured to advance an instrument (e.g., catheter) through a guide sheath. Introducers may be comprised of, e.g., stainless steel. Kits may additionally include one or more transeptal needles or wires, such as mechanical transeptal needles or wires. In some instances, a needle included in the kits is a curved needle (e.g., a Brockenbrough curved needle). In select cases, the kits include a radio frequency transeptal needle or wire.

Accordingly, the preceding merely illustrates the principles of the present disclosure. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein.

What is claimed is:

1. A method comprising:
transeptally delivering an instrument to or beyond the left ventricular outflow tract of a subject via a guide sheath comprising an elongate structure comprising a channel for receiving the instrument running therethrough from a proximal end to a distal end, and wherein the elongate structure comprises:
a first portion which is a fixed curve portion or a deflectable portion; and
a second portion which is a fixed curve portion or a deflectable portion,
wherein the first and second portions are adapted for transeptally delivering the instrument to or beyond the left ventricular outflow tract of the subject.

2. The method according to claim 1, wherein the second portion is distal to the first portion.

3. The method according to claim 2, wherein the first portion is a fixed curve portion.

4. The method according to claim 2, wherein the first portion is a deflectable portion.

5. The method according to any one of claims 2 to 4, wherein the second portion is a deflectable portion.

6. The method according to any one of claims 1 to 5, wherein the first portion is separated by the second portion by a distance of from 0 cm to 50 cm.

7. The method according to any one of claims 1 to 6, wherein the first portion begins at a location along the elongate structure that is at a distance from the distal end ranging from 11 mm to 40 cm.

8. The method according to any one of claims 1 to 7, wherein the first portion ends at a location along the elongate structure that is at a distance from the distal end ranging from 1 cm to 40 cm.

9. The method according to any of claims 1 to 8, wherein the first portion is curved or deflectable to curve at an angle ranging from 5° to 355°.

10. The method according to any one of claims 1 to 9, wherein the second portion begins at a location along the elongate structure that is at a distance from the distal end ranging from 1 mm to 40 cm.

11. The method according to any one of claims 1 to 10, wherein the second portion ends at a location along the elongate structure that is at a distance from the distal end ranging from 0 cm to 39 cm.

12. The method according to any of claims 1 to 11, wherein the second portion is curved or deflectable to curve at an angle ranging from 5° to 355°.

13. The method according to any one of claims 1 to 12, wherein the instrument is a catheter.

14. The method according to claim 13, wherein the instrument is an ablation catheter.

15. The method according to claim 14, wherein the method comprises delivering the ablation catheter to a coronary cusp of the subject.

16. The method according to claim 15, wherein the method comprises ablating the coronary cusp using the ablation catheter.

17. The method according to claim 16, wherein the method treats premature ventricular contractions (PVCs) or ventricular tachycardia of the subject.

18. The method according to any one of claims 1 to 14, wherein the method comprises delivering the instrument to the coronary arteries of the subject.

19. The method according to claim 18, wherein the method comprises injecting contrast dye into the coronary arteries of the subject.

20. The method according to claim 18 or claim 19, wherein the method comprises delivering an angioplasty balloon and/or device used to open and/or expand the lumen of a coronary artery into a coronary artery of the subject.

21. The method according to any one of claims 18 to 20, wherein the method comprises delivering a stent into a coronary artery of the subject.

22. The method according to any one of claims 1 to 14, wherein the method comprises delivering the instrument to the aortic valve of the subject.

23. The method according to claim 22, wherein the method comprises delivering a replacement aortic valve to the subject.

24. The method according to any one of claims 1 to 14, wherein the method comprises delivering the instrument to the ascending aorta of the subject.

25. The method according to claim 24, wherein the method comprises delivering a stent to the ascending aorta of the subject.

26. A guide sheath comprising an elongate structure comprising a channel for receiving an instrument running therethrough from a proximal end to a distal end, and wherein the elongate structure comprises:
a first portion which is a fixed curve portion or a deflectable portion; and
a second portion which is a fixed curve portion or a deflectable portion,
wherein the first and second portions are adapted for transeptally delivering the instrument to or beyond the left ventricular outflow tract of the subject.

27. The guide sheath of claim 26, wherein the second portion is distal to the first portion.

28. The guide sheath of claim 27, wherein the first portion is a fixed curve portion.

29. The guide sheath of claim 27, wherein the first portion is a deflectable portion.

30. The guide sheath of any one of claims 26 to 29, wherein the second portion is a deflectable portion.

31. The guide sheath of any one of claims 26 to 30, wherein the first portion is separated by the second portion by a distance of from 0 cm to 50 cm.

32. The guide sheath of any one of claims 26 to 31, wherein the first portion begins at a location along the elongate structure that is at a distance from the distal end ranging from 11 mm to 40 cm.

33. The guide sheath of any one of claims 26 to 32, wherein the first portion ends at a location along the elongate structure that is at a distance from the distal end ranging from 1 cm to 40 cm.

34. The guide sheath of any of claims 26 to 33, wherein the first portion is curved or deflectable to curve at an angle ranging from 5° to 355°.

35. The guide sheath of any one of claims 26 to 34, wherein the second portion begins at a location along the elongate structure that is at a distance from the distal end ranging from 1 mm to 40 cm.

36. The guide sheath of any one of claims 26 to 35, wherein the second portion ends at a location along the elongate structure that is at a distance from the distal end ranging from 0 cm to 39 cm.

37. The guide sheath of any of claims 26 to 36, wherein the second portion is curved or deflectable to curve at an angle ranging from 5° to 355°.

38. A kit comprising the guide sheath of any one of claims 26 to 37.

39. The kit of claim 38, further comprising instructions for using the guide sheath to transeptally deliver an instrument to or beyond the left ventricular outflow tract of a subject.

40. The kit of claim 38 or claim 39, further comprising a catheter.

41. The kit of claim 40, wherein the catheter is an ablation catheter.

42. The kit of claim 40, wherein the catheter is a balloon catheter.

43. The kit of any of claims 38 to 42, further comprising a balloon.

44. The kit of any of claims 38 to 43, further comprising a catheter introducer.

45. The kit of any of claims 38 to 44, further comprising a mechanical transeptal needle or wire.

46. The kit of any of claims 38 to 44, further comprising a radio frequency transeptal needle or wire.
